(19) United States
(12) Patent Application Publication
(10) Pub. No.: US 2013/0282009 A1
(43) Pub. Date: Oct. 24, 2013

(54) ENDOSCOPIC VEIN HARVESTING SYSTEM

(71) Applicant: CARDICA, INC., Redwood City, CA (US)

(72) Inventor: Bryan D. Knodel, Flagstaff, AZ (US)

(73) Assignee: Cardica, Inc., Redwood City, CA (US)

(21) Appl. No.: 13/864,597

(22) Filed: Apr. 17, 2013

Related U.S. Application Data
(60) Provisional application No. 61/625,845, filed on Apr. 18, 2012.

(51) Int. Cl. A61B 17/00; A61B 18/14

(52) U.S. Cl. CPC A61B 17/00008; A61B 18/1482
USPC 606/47

(57) ABSTRACT

A vein harvesting medical device designed and constructed to harvest a vein in a less invasive surgical procedure. The vein harvesting device includes a handle assembly, a shaft assembly, and an end effector operated by the control features in the handle assembly to gather a targeted vein and remove side branches from the targeted vein for harvesting.

### CROSS-REFERENCE TO RELATED APPLICATION

This application claims priority of U.S. Provisional Application Ser. No. 61/625,845, Attorney Docket No. 265, filed on Apr. 18, 2012, which is hereby incorporated by reference in its entirety.

### FIELD OF THE INVENTION

The invention is generally related to surgical devices; more particularly, to vein harvesting systems and devices.

### BACKGROUND

Typically, a vein may be harvested from a patient to be used for various bypass procedures. One advantage of harvesting a vein from the patient himself/herself is to avoid the potential issue of tissue compatibility and the expenses that may be involved in obtaining one from a donor. A typical vein harvesting procedure involves removing a saphenous vein from the leg of the patient or donor. The procedure requires a long cut starting from the upper thigh down to about the foot area of the leg. Tissue and muscles are moved aside to find and remove the saphenous vein from the leg. As one can appreciate, such a procedure can cause substantial trauma to the leg. As such, a less invasive procedure to harvest a vein for bypass procedures or other applications is desired.

### SUMMARY

A vein harvesting medical device designed and constructed to harvest a vein in a less invasive surgical procedure. The vein harvesting device includes a handle assembly, a shaft assembly, and an end effector operated by the control features in the handle assembly to gather a targeted vein and remove side branches from the targeted vein for harvesting. The end effector includes a tissue separator to separate tissue, a vein capture element with a body that opens and closes to capture a targeted vein, one or more cauterizing element to cauterize side branches connected to the targeted vein, and a knife element to separate the side branches from the targeted vein for harvesting.

A vein harvesting system configured to harvest a vein in a less invasive surgical procedure. The vein harvesting system includes a tissue separator constructed to separate tissue. The vein harvesting system further includes a vein capture element with a body that opens and closes to capture a targeted vein, a cauterizing element to cauterize side branches connected to the targeted vein, and a knife element to separate the side branches from the targeted vein.

A method of harvesting a vein in a less invasive surgical procedure. The method comprises of advancing an end effector with sufficiently small diameter into a patient toward a targeted vein. The method includes manipulating the end effector such that a tissue separator separates tissues to allow the end effector to be advanced toward the targeted vein. Once near the target vein, place a vein capture element near the target vein. The vein capture element includes a body that opens and closes to allow capture of the targeted vein into the body of the vein capture element. After capturing the targeted vein in the body of the vein capture element, retract the vein capture element into the end effector, such that side branches connected to the targeted vein are gathered in one or more side branch slots. The side branches in the side branch slots are cauterized by cauterizing elements placed about the side branch slots. Once the side branches are cauterized, the side branches are cut or separated from the targeted vein by a knife element. The method is repeated until a sufficient amount of the targeted vein has been captured and cleaned of side branches. The captured and cleaned targeted vein is harvested for various surgical operations, e.g., surgical bypass procedures.

### BRIEF DESCRIPTION OF THE DRAWINGS

FIG. 1A and FIG. 1B illustrate one example of an endoscopic vein harvesting device in accordance with one embodiment of the present invention.

FIG. 2A and FIG. 2B illustrate a vein harvesting system of the vein harvesting device in accordance with one embodiment of the present invention.

FIG. 3A and FIG. 3B illustrate tissue and side-branches isolation system of the vein harvesting device in accordance with one embodiment of the present invention.

FIG. 4A and FIG. 4B illustrate tissue and vein side-branches cauterizing and cutting system of the vein harvesting device in accordance with one embodiment of the present invention.

FIG. 5A illustrates the vein harvesting system of the vein harvesting device in accordance with one embodiment of the present invention.

FIG. 5B illustrates an exposed view of the cutting and cauterizing system of the vein harvesting device in accordance with one embodiment of the present invention.

FIG. 5C illustrates another exposed view of the cutting and cauterizing system of the vein harvesting device in accordance with one embodiment of the present invention.

### DETAILED DESCRIPTION

In the following detailed description, specific details are set forth in order to provide a thorough understanding of the present invention. However, it will be understood by those skilled in the art that the present invention may be practiced without these specific details. Alternatively, some of the well-known parts, component, hardware, methods of operations, and procedures may not be described in detail or elaborated so as to avoid obscuring the present invention; but, nevertheless, they are within the spirit and scope of the present invention.

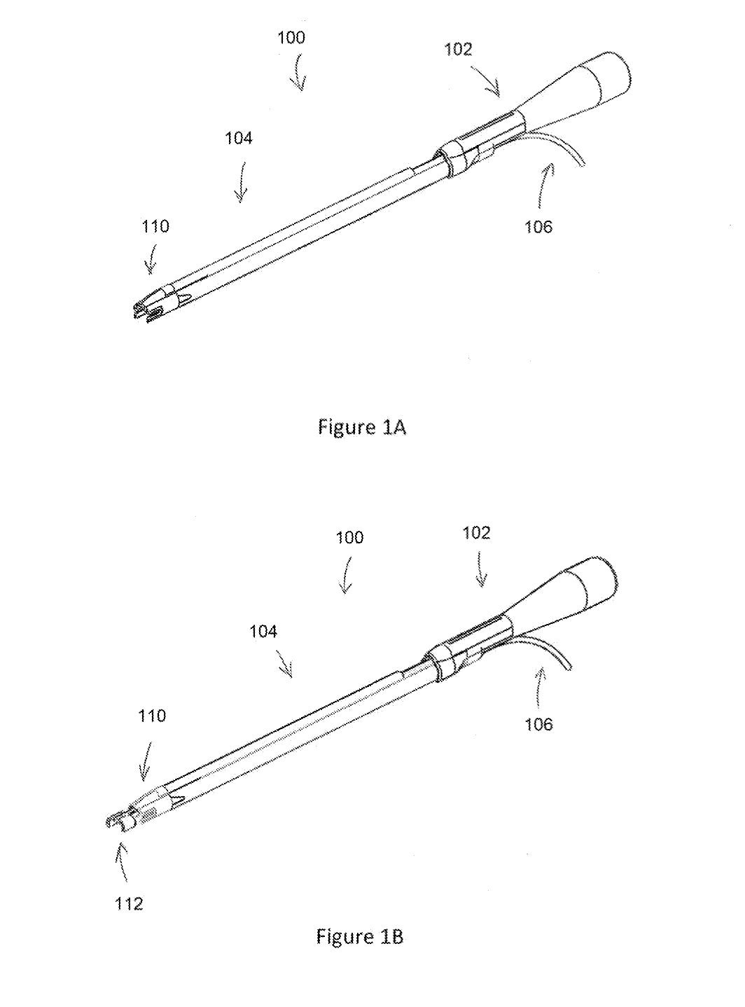

An endoscopic vein harvesting device within the scope and embodiments of the present invention is illustrated in FIG. 1A and FIG. 1B. Typically, a vein may be harvested from a patient to be used for various bypass procedures. One advantage of harvesting a vein from the patient himself/herself is to avoid the potential issue of tissue compatibility and the expenses that may be involved in obtaining one from a donor. A typical vein harvesting procedure involves removing a saphenous vein from the leg of the patient or donor. The procedure requires a long cut starting from the upper thigh down to about the foot area of the leg. Tissue and muscles are moved aside to find and remove the saphenous vein from the leg. As one can appreciate, such a procedure can cause substantial trauma to the leg. As such, a less invasive procedure to harvest a vein for bypass procedures or other applications is desired. The endoscopic vein harvesting device as illustrated in FIG. 1A and FIG. 1B can be used to perform a less invasive procedure to harvest a vein. As illustrated in FIG. 1A and FIG. 1B, the endoscopic vein harvesting device includes a handle assembly 102, a shaft assembly 104, and a control and/or power, pneumatic, or fluid wire 106. In some embodiments, the endoscopic vein harvesting device 100 may be battery operated such that a power wire may not be necessary. In some embodiments, the endoscopic vein harvesting device 100 may be mechanically operated, such that power, pneumatic, or fluid connection(s) may not be necessary. Also, in some embodiments, the endoscopic vein harvesting device may be wirelessly connected to a control unit such that a control wire may not be necessary. The handle assembly 102 may include various operation and/or control mechanisms to operate and/or control various subsystems and components in the shaft assembly 104. The operation and/or control mechanism may include various mechanical and/or electrical subsystems and components, such as mechanical turn-knobs, gears, slides, etc. and electrical sensors, motor, actuators, etc. to operate and/or control the subsystems and components in the shaft assembly 104. Also illustrated in FIG. 1B, the end effector or vein harvesting system 110 of the shaft assembly 104 may include a vein harvesting component or vein capture element 112 for isolating a vein (e.g., the saphenous vein) to be harvested.

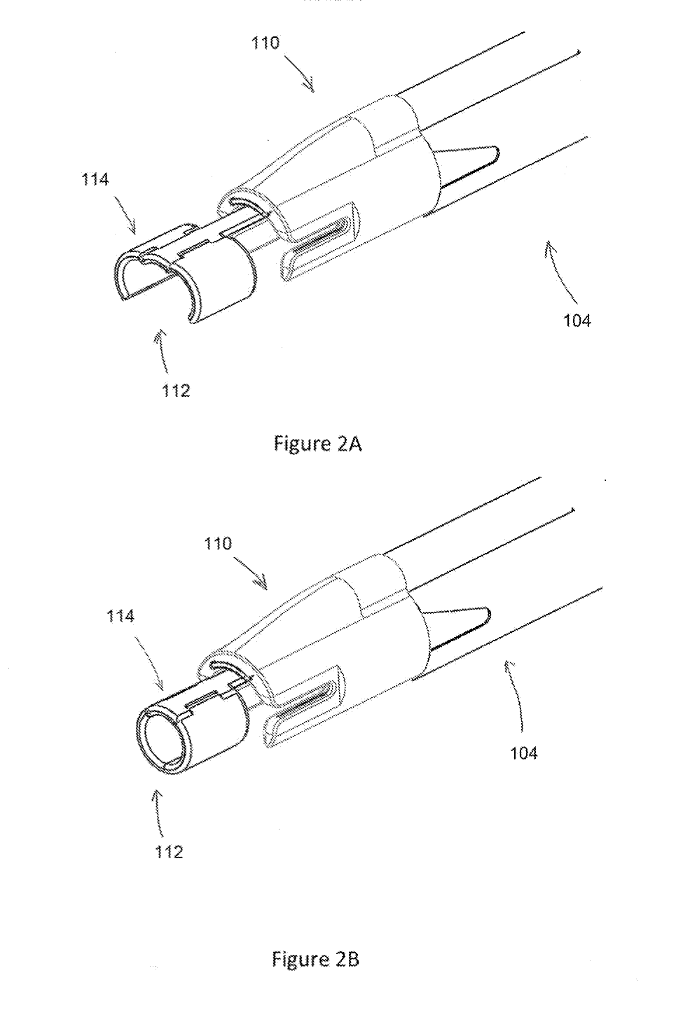

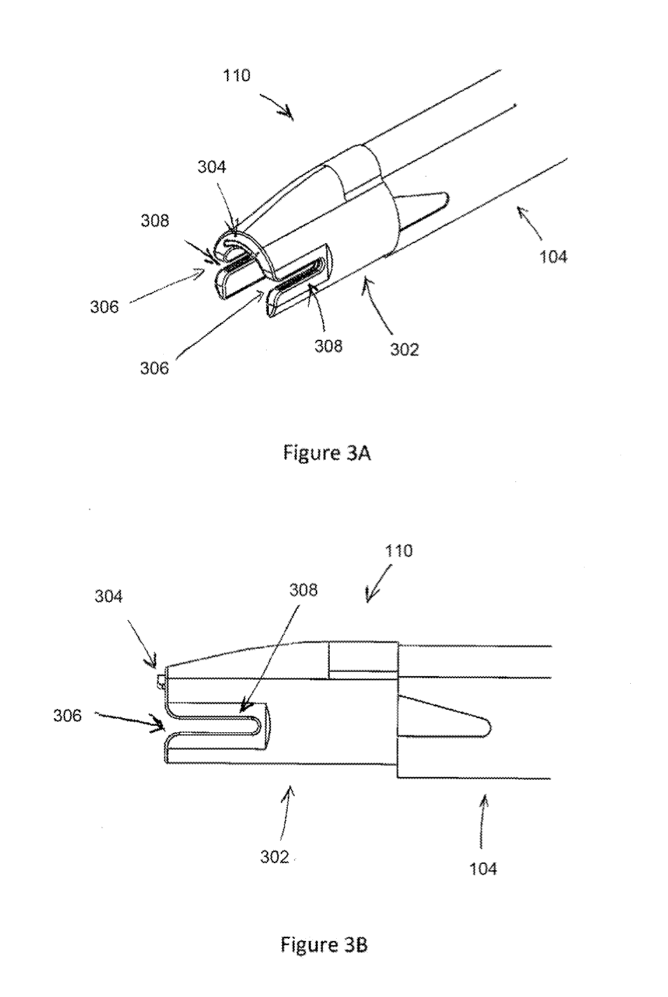

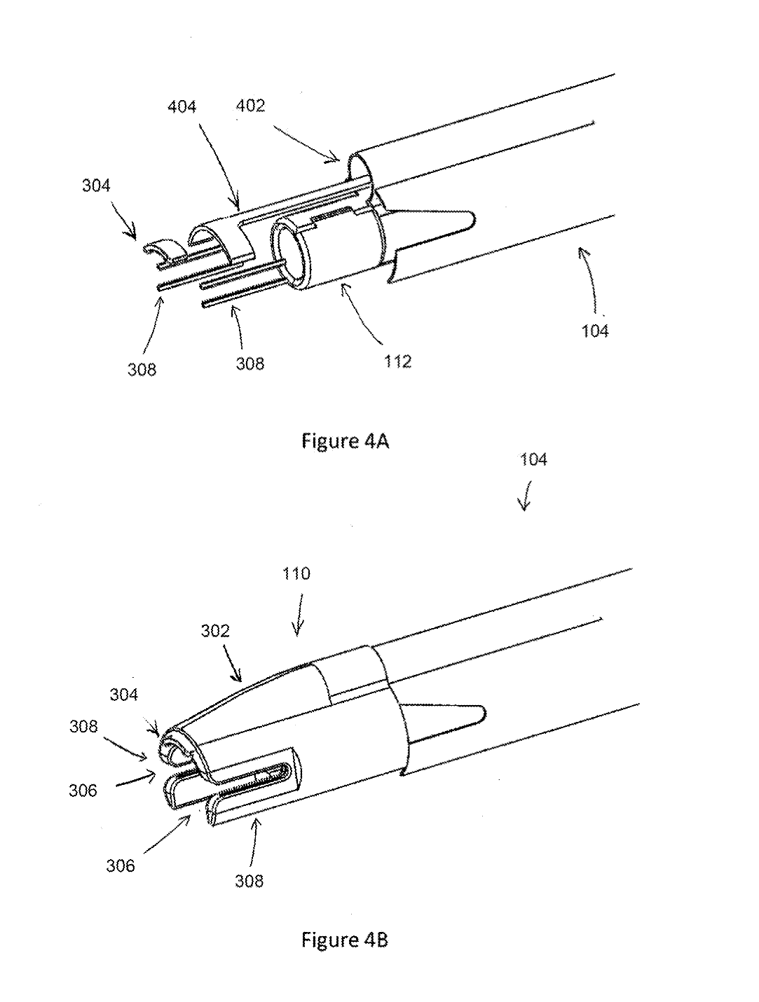

FIG. 2A and FIG. 2B illustrate close-up views of the vein harvesting system 110 of the shaft assembly 104. As illustrated, the vein harvesting system 110 may include a vein capture element 112 with a body 114 that can be opened and closed to capture a targeted vein. As shown in FIG. 2A, the vein capture element 112 may be operated in an open orientation or state with the body 114. During the vein harvesting procedure, the distal portion of the vein harvesting device 100 may be inserted into the leg of a patient through a small cut, port, or trocar. The manipulation, navigation, or advancement of the end effector 110 may be aided by an endoscope, fluoroscopy, or any other suitable device or system that can assist with the operation of the vein harvesting device 100. An endoscope (not shown) may be incorporated into the vein harvesting device 100 and provide various visual accessibilities (e.g., visual, infrared, etc.) at the distal portion of the vein harvesting device through an open channel or lumen 402, as illustrated in FIG. 4A. The end effector 110 or distal portion of the vein harvesting device 100 may be manipulated to find and capture a targeted vein (e.g., saphenous vein) to harvest. To aid with manipulation and navigation of the end effector 110, the shaft portion 104 of the vein harvesting device 100 may be substantially rigid, semi-rigid, substantially flexible, or semi-flexible. In addition, the shaft portion 104 of the vein harvesting device may be actively steerable. The shaft assembly 104 may be actively steered by a subsystem of drive shafts with rack and pinion or various pull-wires, pulleys, and drive gears, etc. Alternatively, the shaft portion 104 may not be actively steerable, and in some embodiments, it may be manipulated and acquire various shapes as necessary to facilitate capture of a targeted vein for harvesting. Once the end effector 110 is manipulated into position to capture a targeted vein, the vein capture element 112 can be extended out of the vein capture system module 110 and into an open position or state with the body 114 to capture the targeted vein, as illustrated in FIG. 2A. The vein capture element 112 can be operated to close the body 114 onto the targeted vein in the extended position or state, as illustrated in FIG. 2B. In this example, the vein capture element body 114 encircles the targeted vein. With the targeted vein captured, to be elaborated further, the vein capture element 112 can be retracted back into the end effector or vein harvesting system 110 for removal of side-branches attached or connected to the targeted vein, as illustrated in FIG. 3A.

FIG. 3A and FIG. 3B illustrate the vein harvesting system 110 of the vein harvesting device 100 in more detail. The vein harvesting system 110 includes a vein harvesting module housing cover 302, a tissue separator 304, side-branch slots 306, and side-branch cauterizing elements 308. As illustrated, the vein harvesting module housing cover 302 may be a substantially open unit. As shown, the housing cover 302 has at least one of its sides (e.g., bottom) open. With one of its sides open, this particular configuration may be especially useful for finding and isolating a targeted vein. For example, once the target vein is located, the opened side (e.g., bottom) can be used to manipulate the housing cover 302 and to some extent the vein capture element 112 as well to capture the targeted vein by lowering the housing cover onto the targeted vein. The vein harvesting system may also include a tissue separator 304 to separate tissue to find the targeted vein. The tissue separator 304 can also be used to separate tissue from the targeted vein once the targeted vein is found. The separator 304 may have a substantially sharp edge to facilitate separation of tissue. The tissue separator 304 may be configured such that it can be extended to increase its effectiveness in separating tissue or retracted when not in use. The tissue separator 304 may be an electrode that ablates and seals tissues as it is operated to separate tissue. The tissue separator 304 may be a mono-polar electrode. Side slots 306 may be used to gather side-branches connected to the targeted vein, and cauterizing elements 308 may be used to cauterize the side-branches before they are separated from the targeted vein. Cauterizing elements 308 may be bi-polar electrodes. In some embodiments, the cauterizing element 308 mono-polar electrodes. In such embodiments, the cauterizing element 308 may not need to work in pairs.

FIG. 4A illustrates some of the vein harvesting elements of the vein harvesting system 110 with the cover 302 exposed. The tissue separator 304 may be a movable component that can be extended or retracted to facilitate separation of tissue. Alternatively, the issue separator 304 may be substantially stationary. The separator 304 may be coupled to the housing cover 302. As illustrated, a cutting element 404 may be used to separate or cut the side-branches that have been gathered within the side slots 306 after the side-branches are cauterized by the cauterizing elements 308. As will be illustrated further, the cutting element 404 may be rotated to cut side-branches. The cutting element 404 may be rotated in substantially circular, semi-circular, or arc motion, passing the side slots 306 to cut or separate the side-branches.

Still referring to FIG. 4A, an open channel or lumen 402 may be used as a pass-through from the proximal portion to the distal portion of the vein harvesting device 100. Various instruments may be passed along from the proximal portion to the distal portion of the vein harvesting device 100. In additional various instruments may be controlled or operated by way of the pass-through path or lumen 402. For example, an endoscopic imaging device (e.g., camera, optic fiber, optic fiber bundle, etc.) may be operated and/or controlled by way of the pass-through 402. In addition, various connection and/or control wires or cables for the operations of the tissue separator 304 and cauterizing elements 308 may be routed along the interior body of the shaft assembly 104 to the proximal portion of the vein harvesting device 100. Alternatively, the various connection and/or control wires or cables for the operations of the tissue separator 304 and cauterizing elements 308 may be routed through the lumen 402. FIG. 4B illustrates the vein harvesting system 110 with the housing cover 302.

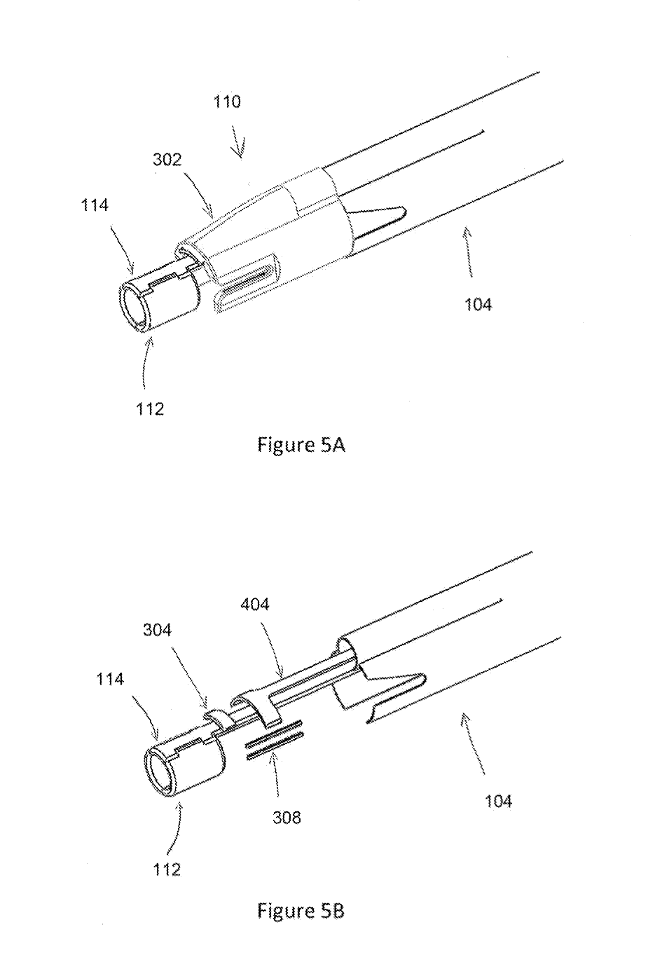

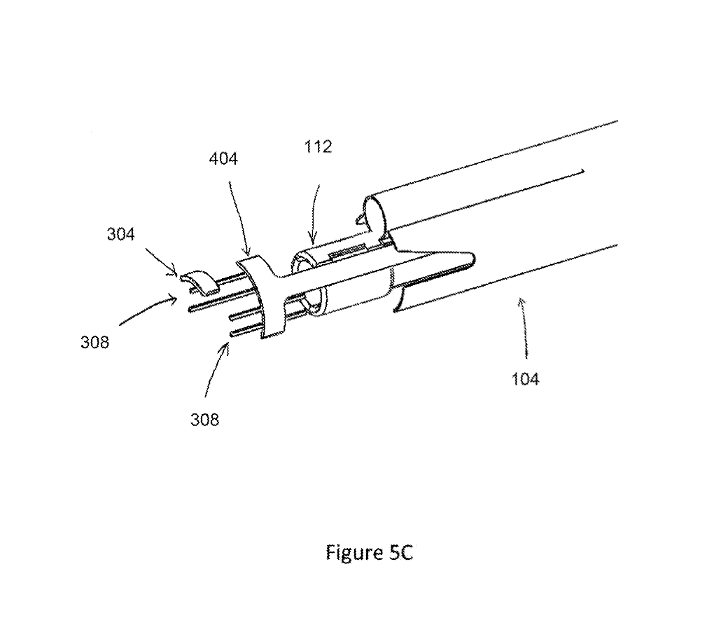

FIG. 5A through 5B illustrate various operational modes of the vein harvesting device 100. FIG. 5A illustrates one example when the end effector 112 has been navigated to or near a targeted vein to be harvested. The tissue separator 304 was used to separate tissue from the targeted vein, and the vein capture element 112 was extended out from the vein harvesting module housing cover 302. The vein capture element 112 opened its body 114 to capture the targeted vein, and then closed its body 114 around a captured vein. FIG. 5B illustrates the various elements and components of the vein harvesting system 110 with the vein capture element 112 extended and the vein harvesting system cover 302 exposed or removed for clarity. As illustrated in FIG. 5C, the vein capture element 112 is retracted into the vein harvesting module 302 where the side-branches connected to the targeted vein would be gathered by the side slots 306. Once the side-branches are gathered in the side slots, the cauterizing elements 308 can be activated to cauterize the side-branches. Once the side-branches are cauterized, knife 404 can be activated to cut the side-branches to separate or shear-off the connections to the targeted vein. The knife element 404 may be activated to travel in a substantially circular, semi-circular, or arc path to cut the side branches. This procedure can be repeated by advancing the end effector 110 of the vein harvesting device 100 down the leg of the patient, separate tissue from the targeted vein by the tissue separator 304, extend and retract the vein capture element 112 to gather side-branches into the side slots 306, cauterize the side-branches with the cauterizing elements 308, cut the cauterized side-branches with the knife element 404, and repeat the steps as necessary until a sufficient length of vein has been captured and cleared of side-branches for removal of the harvested length of vein from the patient's leg.

While the invention has been described in detail, it will be apparent to one skilled in the art that various changes and modifications can be made and equivalents employed, without departing from the scope of the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Words such as “upper,” “lower,” “upward,” “downward”, “front”, “back”, “next to”, and the like are intended for the convenience of the reader and refer to the orientation and motion of parts on the printed page; they do not in any way limit the scope or application of the invention. Topical headings and subheadings, if provided, are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited. The present invention is intended to cover alternatives, modifications, and equivalents that may fall within the spirit and scope of the present invention as defined by the claims.

What is claimed is:

1. A vein harvesting medical device, comprising:
a handle assembly;
a shaft assemble coupled to the handle assembly; and
an end effector coupled to the shaft assembly, comprising:
a tissue separator to separate issue;
a vein capture element including a body that opens and closes to capture a targeted vein for harvesting;
at least one cauterizing element to cauterize side branches connected to the targeted vein; and
a knife element to separate the side branches from the targeted vein.

2. The vein harvesting medical device of claim 1 wherein the end effector further comprises a side branch slot configured to gather side branches for cauterizing and separating side branches from the targeted vein.

3. The vein harvesting medical device of claim 1 wherein the tissue separator includes a substantially shape edge to separator tissue.

4. The vein harvesting medical device of claim 1 wherein the tissue separator is a mono-polar electrode.

5. The vein harvesting medical device of claim 1 wherein the tissue separator is a mono- polar electrode configured to ablate and separate tissue.

6. A vein harvesting system, comprising:
a tissue separator to separate issue;
a vein capture element including a body that opens and closes to capture a targeted vein for harvesting;
a cauterizing element to cauterize side branches connected to the targeted vein; and
a knife element to separate the side branches from the targeted vein.

7. The vein harvesting system of claim 6 further comprising a vein harvesting module housing cover, said vein harvesting module housing cover includes a side branch slot configured to gather side branches for cauterizing and separating side branches from the targeted vein.

8. The vein harvesting system of claim 6 wherein the cauterizing element is a bi-polar electrode.

9. The vein harvesting system of claim 6 wherein the knife element is operated in a substantially semi-circular path to cut or separate side branches from the targeted vein for harvesting.

10. A method of harvesting a vein in a less invasive surgical procedure, comprising:
advancing an end effector toward a targeted vein in a patient;
separating tissues to allow the end effector to be advanced toward the targeted vein;
placing a vein capture element near the targeted vein;
capturing the targeted vein in a body of the vein capture element;
cauterizing side branches connected to the targeted vein; and
separating cauterized side branches from the targeted vein.
